(19) United States
(12) Patent Application Publication
(10) Pub. No.: US 2015/0223696 A1
(43) Pub. Date: Aug. 13, 2015

(54) METHOD FOR EVALUATING AGING, AND DEVICE FOR EVALUATING AGING

(71) Applicant: Sharp Kabushiki Kaisha, Osaka-shi, Osaka (JP)

(72) Inventor: Mikihiro Yamanaka, Osaka-shi (JP)

(21) Appl. No.: 14/424,154

(22) PCT Filed: Aug. 30, 2013
(86) PCT No.: PCT/JP2013/073281

§ 371 Date: Feb. 26, 2015

(30) Foreign Application Priority Data
Sep. 19, 2012 (JP) 2012-206349

(51) Int. Cl. A61B 5/00

(52) U.S. Cl. CPC A61B 5/0071; A61B 5/4869; A61B 5/6826

(57) ABSTRACT

A method for evaluating aging of the present invention, evaluates a progress degree of aging, based on a fluorescent material which increases in a body, along with the aging of a living being. Moreover, a device for evaluating aging of the present invention, includes an aging determining unit that outputs a determination result indicating the progress degree of the aging, by irradiating the living being with excitation light having a wavelength of a predetermined range, and by performing a predetermined calculation using a fluorescence intensity of a fluorescence spectrum emitted from a fluorescent material which is characteristic in an old stage in comparison with an immature stage, and a controlling unit for a display device to display the determination result which is output by the aging determining unit. According to the present invention, it is possible to simply evaluate the aging on the spot without using a genetic analysis or the like.

### TECHNICAL FIELD

The present invention relates to a method for evaluating aging and a device for evaluating aging, which evaluate aging of a human being.

### BACKGROUND ART

There has been known a technology for evaluating aging. For example, in PTL 1, it is disclosed a method for evaluating skin including a step of measuring expression of at least one gene that is selected from a group consisting of an E-cadherin gene, a T-cadherin (H-cadherin) gene, a Poliovirus receptor gene, an Integrin beta-1 gene, a Laminin alpha 3 gene, a Jagged 1 gene, a Delta-like 1 gene and a Keratin 10 gene, in a sample originating from a living body which is taken from a subject, and a step of evaluating a skin aging degree of the subject, based on an expression level of the gene of a measurement target.

### CITATION LIST

### Patent Literature

PTL 1: Japanese Unexamined Patent Application Publication No. 2010-115131 (published on May 27, 2010)

### SUMMARY OF INVENTION

### Technical Problem

However, the related art as described above, is a complicated technology such as a genetic analysis, and needs preprocessing such as an adjustment of the sample before performing the genetic analysis. Moreover, in the genetic analysis, a high-priced analysis device is needed. Hence, it is not possible to measure, and evaluate the aging degree on the spot.

The present invention is made for solving the above problem, and an object of the present invention is to provide a method for evaluating aging which can simply evaluate the aging on the spot without using the genetic analysis or the like.

### Solution to Problem

In order to solve the above problem, a method for evaluating aging according to one aspect of the present invention, evaluates a progress degree of aging, based on a fluorescent material which increases in a body, along with the aging of a living being.

### Advantageous Effects of Invention

According to one aspect of the present invention, an effect that the aging can be simply evaluated, is achieved.

### BRIEF DESCRIPTION OF DRAWINGS

FIG. 1 is a diagram illustrating an example of a configuration of a device for evaluating aging according to an embodiment.

FIG. 2 is a diagram illustrating a relationship between a wavelength of excitation light and a fluorescence spectrum, at the time of irradiating an aging model living being with the excitation light.

FIG. 3 is a graph illustrating an increase of a fluorescence intensity along with aging of the aging model living being.

FIG. 4 is a diagram illustrating a 17-day-old structure analysis result.

FIG. 5 is a diagram illustrating the fluorescence spectrum that is obtained by saccharifying protein which increases along with the aging.

FIG. 6 is a diagram illustrating the fluorescence spectrum which is obtained at the time of giving different feeds to each of two 7-day-old aging model living beings.

FIG. 7 is a diagram illustrating an example of a configuration of a device for evaluating aging according to Modification Example 1.

FIG. 8 is a diagram illustrating an example of a configuration of a fingertip measuring device.

FIG. 9 is a diagram illustrating an example of a configuration of a device for evaluating aging according to Modification Example 3.

FIG. 10 is a diagram illustrating a measuring probe which is included in a device for evaluating aging according to Modification Example 4.

FIG. 11 is a block diagram illustrating an example of a configuration of a control device.

### DESCRIPTION OF EMBODIMENTS

Embodiments of the present invention will be described, with reference to the drawings. In the following description, the same reference signs are attached to the same members. Names thereof and functions thereof are the same. Therefore, the detailed description thereof will not be repeated.

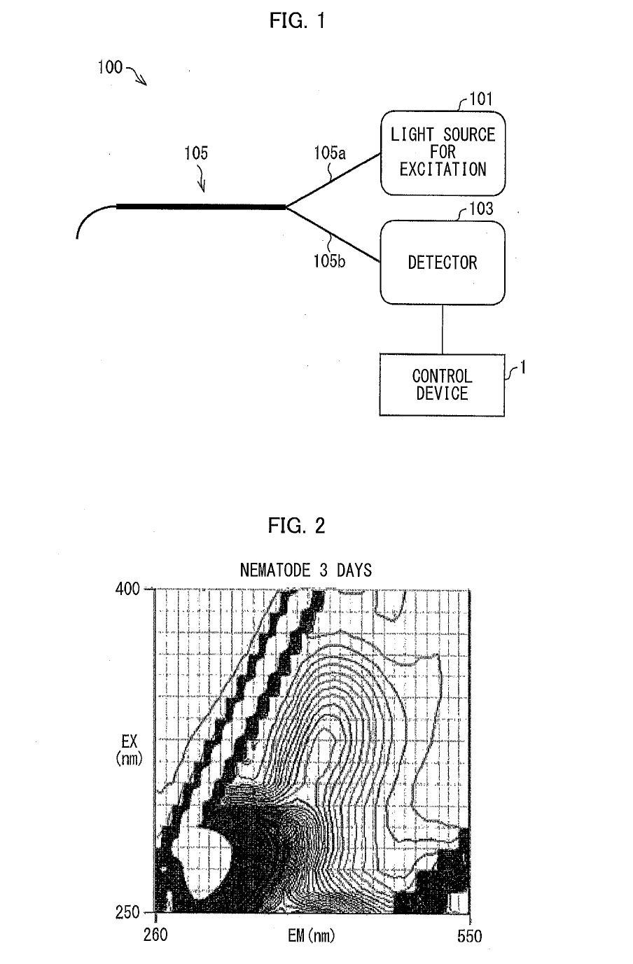

FIG. 1 is a diagram illustrating an example of a configuration of a device for evaluating aging according to an embodiment. As illustrated in FIG. 1, a device for evaluating aging 100 is a device that evaluates the aging of a human being, and includes a light source for excitation 101, a detector 103, a measuring probe 105, and a control device 1.

In the measuring probe 105, two types of an optical fiber for entrance 105a and an optical fiber for exit 105b are coaxially arranged. The light source for excitation 101 is attached to one end of the optical fiber for entrance 105a, and excitation light which is emitted from the light source for excitation 101, is transmitted, and exits from the other end of the optical fiber for entrance 105a. Furthermore, the light source for excitation 101 is a light source that emits the excitation light having a wavelength of 325 nm, and may be, for example, a bulb type such as a halogen light source or a xenon light source, an LED (manufactured by DOWA Electronics Materials Co., Ltd., or the like), an LD or the like.

For example, the end portion of the exit side of the optical fiber for entrance 105a is directed toward an evaluation target of a living being. Hereby, it is possible to irradiate the evaluation target with the excitation light which is emitted from the light source for excitation 101. The present applicant pays attention to a case that an accumulation quantity of a fluorescent material which emits fluorescence by the excitation light, increases in a body while the aging of the living being progresses. The fluorescent material is a material that emits the fluorescence by being excited by the light having a specific wavelength. The light which excites the fluorescence, is referred to as the excitation light.

The optical fiber for exit 105b is an optical fiber of which the end portion of an entrance side is the end portion of the exit side of the optical fiber for entrance, and the fluorescence emitted from the evaluation target enters from the end portion of the entrance side. In the optical fiber for exit 105b, the end portion of the exit side of the fluorescence is connected to the detector 103.

The detector 103 is a device that detects the fluorescence, and may be, for example, a CCD detector IL X511B (manufactured by SONY Corporation), a photodetector; PD (Si PIN photodiode manufactured by Hamamatsu Photonics), a CMOS image sensor, a photomultiplier tube (PMT), a channel TRON detector, or the like. The detector 103 detects the fluorescence which is transmitted by the optical fiber for exit 105b, and outputs a detection result to the control device 1.

The control device 1 may be a device which can perform a luminance adjustment of the light source for excitation 101, a switching control of irradiation or non-irradiation, storage of data, a display of the data, an analysis of the data, or the like, and for example, the control device 1 is a personal computer. The control device 1 displays a fluorescence spectrum on a monitor, as the detection result which is input from the detector 103.

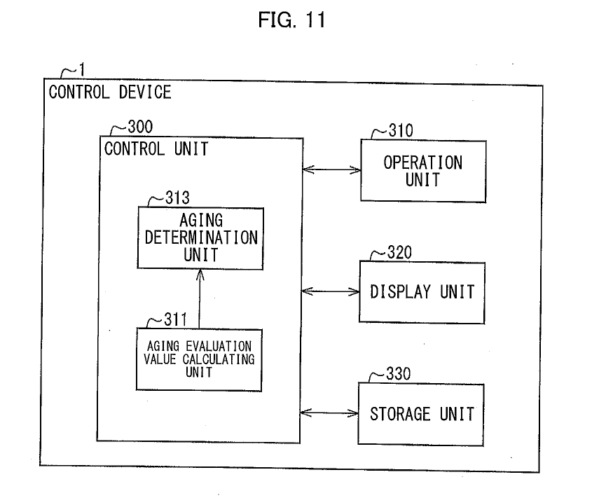

FIG. 11 is a block diagram illustrating an example of the configuration of the control device. As illustrated in FIG. 11, the control device 1 includes a control unit 300, an operation unit 310, a display unit 320, and a storage unit 330. For example, if the control device 1 is the personal computer, since a hardware configuration thereof is well-known, here, the detailed description will not be repeated. The control unit 300 which is included in the control device 1, includes an aging evaluation value calculating unit (means for calculating an aging evaluation value) 311, and an aging determination unit (means for determining aging) 313.

The aging evaluation value calculating unit 311 calculates an aging evaluation value indicating a progress degree of the aging, and outputs the calculated evaluation value to the aging determination unit 313. Specifically, a value indicating a size degree of a reference fluorescence intensity with respect to a measured fluorescence intensity, is calculated as an aging evaluation value, based on the measured fluorescence intensity, and the reference fluorescence intensity which is stored in the storage unit 330. The measured fluorescence intensity is the fluorescence intensity which is input from the detector 103. The aging evaluation value may be a difference or a ratio of the measured fluorescence intensity with respect to the reference fluorescence intensity.

The aging determination unit 313 determines the progress degree of the aging, based on the aging measurement value which is calculated by the aging evaluation value calculating unit 311, and aging evaluation data which is stored in the storage unit 330, and controls to cause the display unit 320 to display a determination result. The aging evaluation data is the data that sets up the progress degrees of the aging by the aging evaluation values, and the progress degrees of the aging are associated with each of a plurality of aging evaluation value ranges.

The aging determination unit 313 refers to the aging evaluation data which is stored in the storage unit 330, and specifies the range including the aging evaluation value which is calculated by the aging evaluation calculating unit 311 among the plurality of ranges indicated by the aging evaluation data. The aging determination unit 313 outputs the progress degree of the aging corresponding to the range which is specified in the aging evaluation data onto the display unit 320, as a determination result.

### <Method for Evaluating Aging>

Here, a method for evaluating aging using the device for evaluating aging 100, will be described. In preparation for the evaluating of the aging, the fluorescence spectrum which becomes an aging marker, is specified. Specifically, using a commercial product (FL-4500, fluorescence spectrophotometer manufactured by Hitachi High-Technologies Corporation), the light which is emitted from the light source for excitation is adjusted in the range of 250 nm to 400 nm, and an old stage and an immature stage of the aging model living being are irradiated with the adjusted light. Hereby, particularly in the old stage of the aging model living being, the characteristic fluorescence spectrum can be confirmed. Furthermore, the used aging model living being is a nematode, and it is generally known that the nematode can be applied to an aging model of the human being.

FIG. 2 is a diagram illustrating a relationship between the wavelength of the excitation light and the fluorescence spectrum, at the time of irradiating the aging model living being with the excitation light. A vertical axis indicates the wavelength of the excitation light which is from 250 nm to 400 nm, and a horizontal axis indicates the fluorescence spectrum. In FIG. 2, the fluorescence spectrum is plotted depending on the wavelength of the excitation light. FIG. 2 indicates the spectrum where the fluorescence depending on the specific wavelength of the excitation light is emitted at the spot of which density is high in a bird's-eye view, and by FIG. 2, it is confirmed that the fluorescence spectrum (Em) of 420 nm is present when being excited with the excitation light (Ex) having the wavelength of 325 nm. Hence, the fluorescent material which emits Em: 420 nm by being excited with Ex: 325 nm, is assumed to be a material that is accumulated in the body along with the aging, and a test using Ex: 325 nm and Em: 420 nm as an aging marker, is performed.

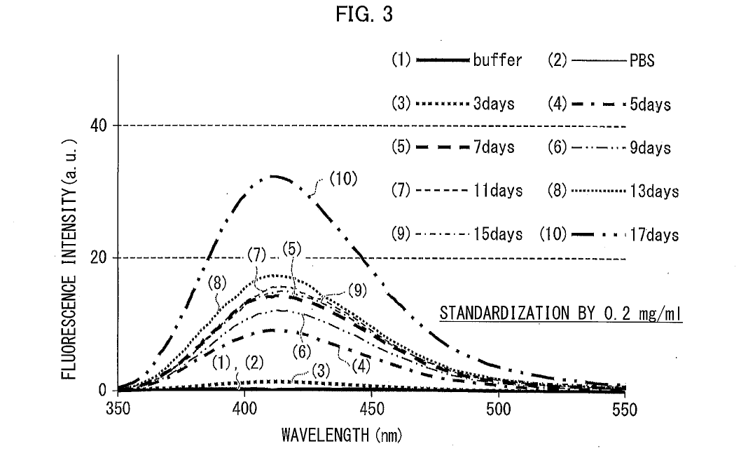

FIG. 3 is a graph illustrating an increase of the fluorescence intensity along with the aging of the aging model living being. The horizontal axis indicates the wavelength, and the vertical axis indicates the fluorescence intensity. As illustrated in FIG. 3, a plurality of curves (1) to (9), respectively indicate the fluorescence intensities of the aging model living being in different measuring periods. Specifically, every 2 days from 3-day-old after birth, each age till 17-day-old at which a lifespan ends is used as a measuring period, and the fluorescence intensity is measured, and thereby, the curve is obtained.

Fluorescent markers are Ex: 325 nm, and Em: 420 nm described above. Moreover, in order to standardize a concentration of protein, a quantitative determination of the protein concentration is performed by a Bradford method, and the fluorescence intensity is measured in the measuring period, as an equivalent quantity of 0.2 mg/ml. As a result, the fluorescence intensity indicates the fluorescence which increases from 3-day-old after birth till 5-day-old, as illustrated in the drawing. Moreover, from 7-day-old till 13-day-old, it is observed to be the very similar fluorescent intensity. Since the aging model living being is at the spawning season from 5-day-old, it is suggested that production of the fluorescent material occurs in the body, along with a change of a growth factor which is gradually generated in the body for this period. Still more, at 17-day-old, the lifespan of the aging model living being ends, and thus, the high fluorescence intensity is indicated in comparison with the curve till 15-day-old. Hence, at 17-day-old, it is expected that a possibility of receiving oxidative stress is high.

From the result of FIG. 3, in comparison with 3-day-old and 17-day-old, since it is possible to expect that the fluorescent material of which the fluorescence intensity is high at 17-day-old is produced in the body, the fluorescent material is identified. Specifically, a structure of the fluorescent material is analyzed using an analysis device which is referred to as LC-MALDI (LC-MALDI QSTAR 5800 manufactured by AB Sciex company). By liquid chromatography, structure analysis results of 3-day-old and 17-day-old are compared, and thereby, it is confirmed that the fluorescent material of which the fluorescence intensity clearly increases at 17-day-old, and fractionation with respect to the fluorescent material is performed using a column, and the structure of the fluorescent material is analyzed.

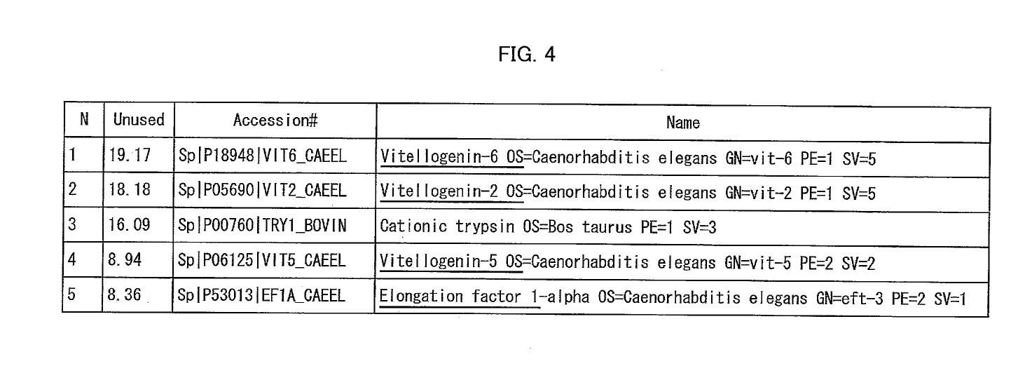

By a peak which is identified from a protein database (public database Swiss Prot), an Elongation factor and Vitellogenin-2, Vitellogenin-5, and Vitellogenin-6 are confirmed at 17-day-old (see FIG. 4). It is known that the Elongation factor is oxidized along with the aging, and it is reported that the Vitellogenins are precursor proteins of egg yolk hormones, and are accumulated in an intestinal tract. The material of which all proteins are AGEd along with the aging, emits the fluorescence.

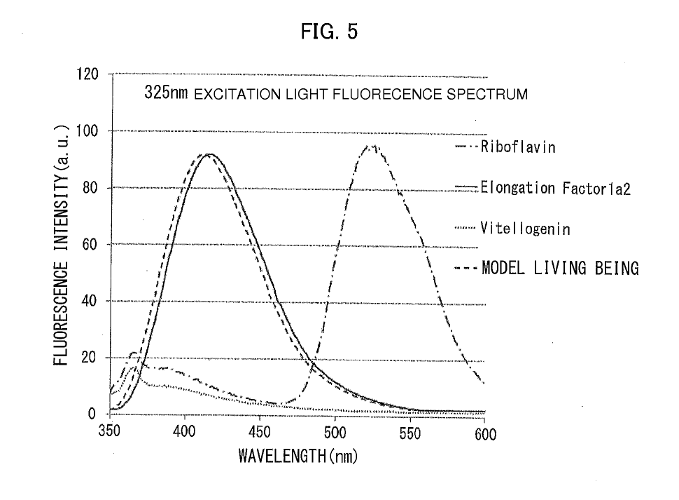

FIG. 5 is a diagram illustrating the fluorescence spectrum that is obtained by saccharifying the protein which increases along with the aging. The wavelength of the excitation light (Ex) is 325 nm. Here, as protein which increases along with the aging, a Riboflavin, the Elongation factor, and the vitellogenin are targeted. The proteins are mixed with a glucose solution, and are incubated for 10 days at 35° C., and thereby, the proteins are saccharified. A plurality of curves illustrated in FIG. 5, indicate the fluorescence spectrum of saccharified materials of the proteins.

As illustrated in FIG. 5, it is found out that the fluorescence intensity of the fluorescence spectrum (Em) of the saccharified material of the Elongation factor is high at 420 nm. That is, it is confirmed that the saccharified material of the Elongation factor has a high correlation with the fluorescent markers of Ex: 325 nm and Em: 420 nm. As a result, it is clear that the fluorescent markers of Ex: 325 nm and Em: 420 nm are the fluorescent materials of which main factors are the Elongation factors.

Furthermore, there is a possibility that the vitellogenin is the aging marker in the structure analysis, but it is confirmed that the fluorescence intensity is low under an excitation condition of Ex: 325 nm, and it is confirmed that the fluorescence is not emitted as a molecular structure, and thus, the vitellogenin is excluded from the target of the fluorescent markers of Ex: 325 nm and Em: 420 nm.

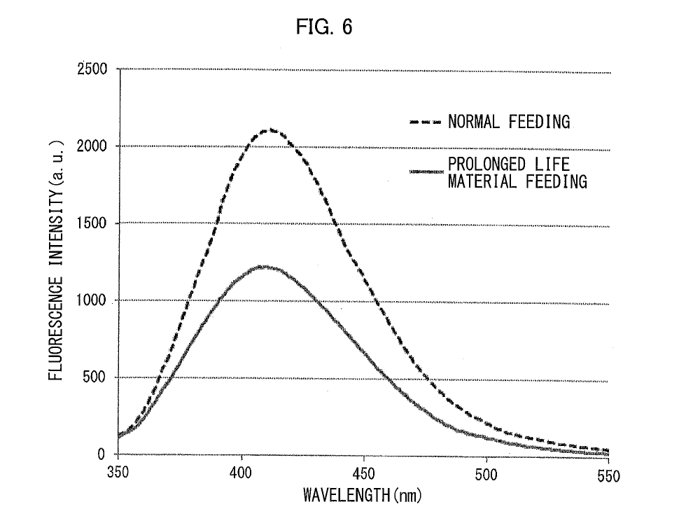

Next, using the method for evaluating aging as described above, the fluorescence spectrum which is obtained at the time of giving different feeds to each of two aging model living beings at the same day-old, is measured. FIG. 6 is a diagram illustrating the fluorescence spectrum which is obtained at the time of giving the different feeds to each of two 7-day-old aging model living beings. The wavelength of the excitation light (Ex) is 325 nm. A normal feed is given to A which is one of the aging model living beings, and a feed including a prolonged life material is given to B which is the other of the aging model living beings. The prolonged life material is vitamin C.

As illustrated in FIG. 6, in comparison with the aging model living being A, the fluorescence intensity of the aging model living being B is lowered. Hereby, it is found out that the Elongation factor which increases along with the aging can be lowered in the aging model living being B. Hence, it is possible to determine that the giving of the prolonged life material is suitable as an effect of suppressing the increase of the Elongation factor, and it is proven that the method for evaluating aging is correct. Furthermore, the method for evaluating aging can obtain a guideline on a prolonged life effect.

Moreover, since the aging model living being can be applied to the aging model of the human being, it is possible to obtain the guideline on the prolonged life effect of the human being. Furthermore, as a method for promoting the prolonged life of the human being, it is clear to allow screenings of the prolonged life by a method for suppressing oxidation of the protein by giving coenzymes such as polyphenols, vitamins, carotenoids, glutathione and ubiquinol, uric acid or lipoic acid as an antioxidant, the prolonged life by exercise, and the prolonged life by suppressing stress, in addition to the method for giving the prolonged life material.

### Modification Example 1

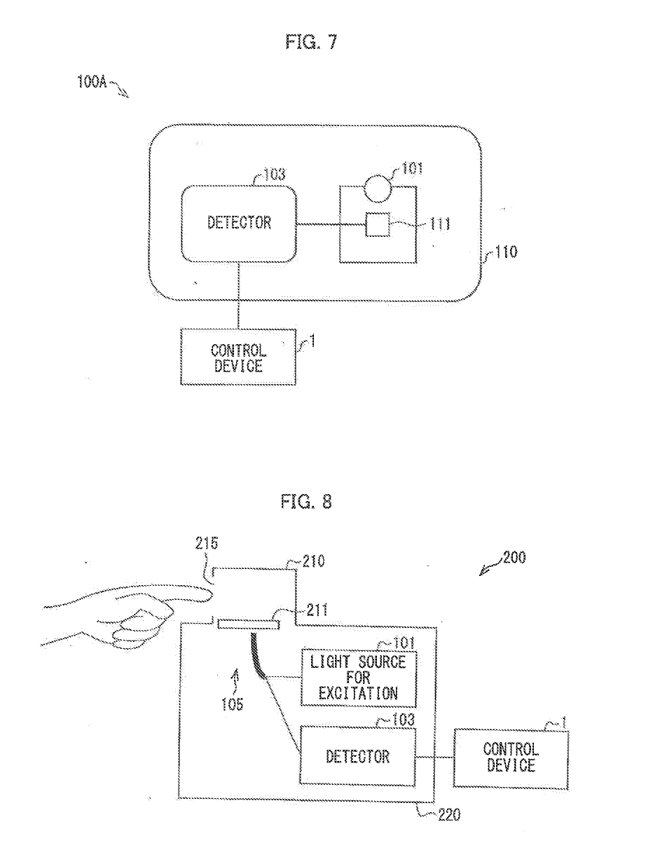

A device for evaluating aging according to Modification Example 1, is configured to detect the fluorescence when the evaluation target is a solution-shaped target. FIG. 7 is a diagram illustrating an example of a configuration of the device for evaluating aging according to Modification Example 1. As illustrated in FIG. 7, a device for evaluating aging 100A includes the control device 1, and a measuring unit 110. Within a case of the measuring unit 110, the light source for excitation 101, the detector 103, and a cell holder 111 are placed. The light source for excitation 101 irradiates toward the cell holder 111 with the excitation light. A transparent container into which the solution-shaped evaluation target is put, is placed in the cell holder 111, and the excitation light which is emitted from the light source for excitation 101 goes through the solution-shaped evaluation target, and enters the detector 103.

### Modification Example 2

Modification Example 2 is configured to include a fingertip measuring device in the device for evaluating aging 100 according to the embodiment. Since other configurations and other functions are the same as the device for evaluating aging 100, here, the description will not be repeated.

FIG. 8 is a diagram illustrating an example of a configuration of the fingertip measuring device. As illustrated in FIG. 8, a fingertip measuring device 200 has a fingertip insertion unit 210, and a measuring member placement unit 220. The measuring member placement unit 220 has a measuring stand 211 which comes into contact with a fingertip, in a portion which is communicated with the fingertip insertion unit 210. In the measuring stand 211, a hole of 5 mmφ to 10 mmφ is arranged in order to pick out the excitation light from the measuring probe 105 described later, and a quartz cover glass (not illustrated) is installed in the hole. The fingertip insertion unit 210 has an insertion hole 215 and a space for inserting the fingertip.

Additionally, in the measuring member placement unit 220, the end portion which is the exit side of the optical fiber for entrance 105a as well as the entrance side of the fiber for exit 105b in the measuring probe 105, is placed in a state of being directed toward the cover glass of the measuring stand 211. Hereby, since the fingertip which is mounted on the measuring stand 211 by being inserted due to the fingertip insertion unit 210, can be irradiated with the excitation light, as well as the light which is emitted from the fingertip by the irradiated excitation light, can be guided to the detector 103, it is possible to detect the fluorescence.

Moreover, when an user inserts a finger into the fingertip insertion unit 201, due to presence of the measuring stand 211, since it is prevented that the finger pushes a tip of the measuring probe 105, it is possible to maintain a distance relationship between the end portion which is the entrance side of the fiber for exit 105b, and the target (fingertip) constant. Furthermore, the measuring stand 211 is set to be larger than a diameter of the end portion which is the entrance side of the fiber for exit 105b. Hereby, using an infrared camera, it is possible to measure a position of a blood vessel of the fingertip.

The fingertip is a spot where the AGEd materials are likely to be piled. Therefore, the spot for measuring the fluorescence which is emitted by the AGEd material, is the fingertip, and thereby, it is possible to enhance measuring accuracy. In addition thereto, since melanin is not present in the fingertip, there is no need to pay attention to absorption of the excitation light due to the melanin at the time of transdermal fluorescence measurement. That is, by removing an influence of suntan or an influence due to the differences of races (which can be also measured in the colored race, and the white race), it is possible to evaluate the progress degree of the aging.

### Modification Example 3

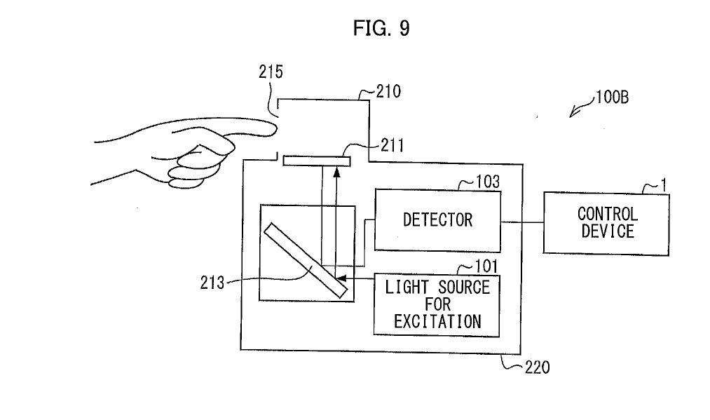

A device for evaluating aging according to Modification Example 3, is configured to irradiate toward the fingertip insertion unit 210 with the excitation light, and to detect the fluorescence, without using the measuring probe 105. Points which are different from the fingertip measuring device 200 according to Modification Example 2, will be mainly described. FIG. 9 is a diagram illustrating an example of a configuration of a device for evaluating aging according to Modification Example 3. As illustrated in FIG. 9, the light source for excitation 101 and the detector 103 which are included in a device for evaluating aging 100B, are placed in the measuring member placement unit 220, and a reflective mirror 213 is placed at a predetermined angle with respect to a horizontal direction, below the fingertip insertion unit 210.

The reflective mirror 213 reflects the excitation light which is emitted from the light source for excitation 101, and the light exits toward the space of the fingertip insertion unit 210. Hence, by inserting the fingertip into the fingertip insertion unit 210, it is possible to irradiate the fingertip with the excitation light. Moreover, the fluorescence which is emitted by irradiating the fingertip with the excitation light, exits toward the space of the measuring member placement unit 220, and is reflected by the reflective mirror 213, and enters the detector 103.

### Modification Example 4

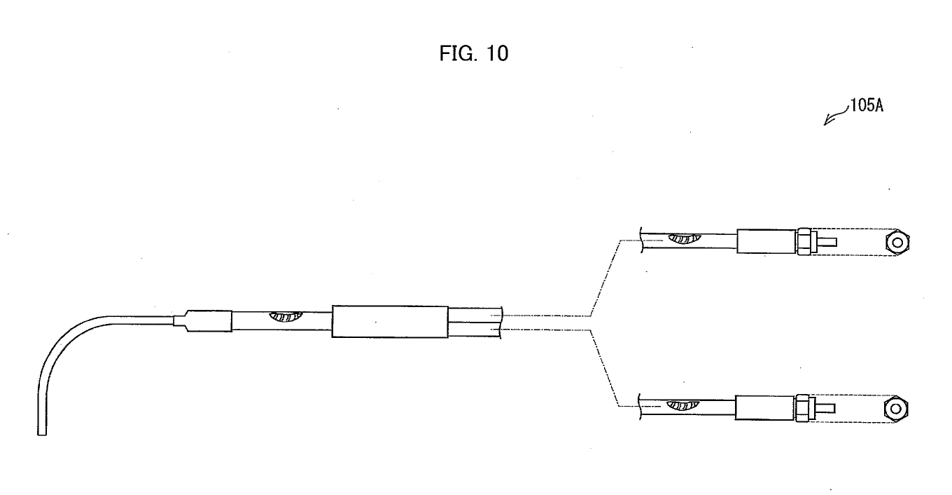

As illustrated in FIG. 10, since a device for evaluating aging according to Modification Example 4 is configured to include a measuring probe 105A of which the tip is bent by 90 degrees, and other configurations are the same as the device for evaluating aging 100 according to the embodiment, here, the description will not be repeated. Since a post of the measuring probe 105A is fixed to a fixing shaft, the excitation light is basically generated downwards. Hence, it is possible to easily make the measurement of the aging model living being in an incubation container. Moreover, since the tip of the measuring probe 105A is bent by 90 degrees, a concern that the light directly enters measurer's eyes, is remarkably reduced.

### SUMMARY

As described above, the method for evaluating aging according to one aspect of the present invention, includes evaluating the progress degree of the aging, based on the fluorescent material which increases in the body, along with the aging of the living being.

According to the method described above, since the fluorescent material is the evaluation target of the aging, there is no need to include a high-priced device and a complicated process such as a genetic analysis. Therefore, since it is sufficient only by specifying the fluorescent material which increases in the body along with the aging, the evaluation of the aging is simple, and it is possible to evaluate the aging on the spot. Consequently, it is possible to simply evaluate the aging on the spot without using the genetic analysis or the like. Moreover, it is considered application of developing a device which measures the aging of skin due to the saccharification, and making visualization of the effects of anti-aging cosmetics or anti-aging supplements.

The method for evaluating aging according to one aspect of the present invention, includes measuring the fluorescence intensity of the fluorescence spectrum emitted from the fluorescent material which is characteristic in the old stage in comparison with the immature stage, by irradiating the living being with the excitation light having the wavelength of a predetermined range, and thereby, the progress degree of the aging is evaluated.

In the method for evaluating aging according to one aspect of the present invention, it is preferable that the wavelength of the excitation light is in a range of 305 nm to 365 nm.

In the method for evaluating aging according to one aspect of the present invention, it is preferable that the fluorescence spectrum where the fluorescent material is at the maximum fluorescence intensity, is included in a range of 400 nm to 470 nm±full width at half maximum.

Furthermore, in the embodiment, and Modification Examples 1 to 4, in order to make measurement conditions uniform, a control circuit may be added so that an output of the excitation light is kept constant at all times. Since the commercial fluorescence spectrophotometer is a large scale and is not versatile, space-wise and cost-wise limits may occur on the measurement.

The present invention is not limited to the embodiments described above, and may be variously altered within the scope which is indicated in the claims. The embodiments that are obtained by appropriately combining the technical means which are respectively disclosed in the different embodiments, are also included in the technical scope of the present invention.

### Reference Signs List

1 CONTROL DEVICE

100, 100A, 100B DEVICE FOR EVALUATING AGING

101 LIGHT SOURCE FOR EXCITATION

103 DETECTOR

105, 105A MEASURING PROBE

105a OPTICAL FIBER FOR ENTRANCE

105b OPTICAL FIBER FOR EXIT

110 MEASURING UNIT

111 CELL HOLDER

200 FINGERTIP MEASURING DEVICE

210 FINGERTIP INSERTION UNIT

211 MEASURING STAND

213 REFLECTIVE MIRROR

215 INSERTION HOLE

220 MEASURING MEMBER PLACEMENT UNIT

300 CONTROL UNIT

311 AGING EVALUATION VALUE CALCULATING UNIT (MEANS FOR CALCULATING AN AGING EVALUATION VALUE)

313 AGING DETERMINATION UNIT (MEANS FOR DETERMINING AGING)

1. A method for evaluating aging, comprising:
evaluating a progress degree of aging, based on a fluorescent material which increases in a body, along with the aging of a living being.

2. The method for evaluating aging according to claim 1, further comprising:
measuring a fluorescence intensity of a fluorescence spectrum emitted from the fluorescent material which is characteristic in an old stage in comparison with an immature stage, by irradiating the living being with excitation light having a wavelength of a predetermined range.

3. The method for evaluating aging according to claim 2,
wherein the wavelength of the excitation light is in a range of 305 nm to 365 nm.

4. The method for evaluating aging according to claim 3,
wherein the fluorescence spectrum where the fluorescent material is at the maximum fluorescence intensity, is included in a range of 400 nm to 470 nm±full width at half maximum.

5. A device for evaluating aging, which evaluates aging by using the method for evaluating aging according to claim 1.

6. The device for evaluating aging according to claim 5, comprising:
an aging determining unit that outputs a determination result indicating a progress degree of aging, by irradiating a living being with excitation light having a wavelength of a predetermined range, and by performing a predetermined calculation using a fluorescence intensity of a fluorescence spectrum emitted from a fluorescent material which is characteristic in an old stage in comparison with an immature stage; and
a display controlling unit that causes a display unit to display the determination result which is output by the aging determining unit.

7. The device for evaluating aging according to claim 5, further comprising:
a fingertip insertion unit into which a fingertip is inserted;
a light source unit that irradiates the fingertip which is inserted into the fingertip insertion unit, with the excitation light; and
a detection unit that detects the fluorescence spectrum which is generated by the irradiation of the excitation light.
